(19) United States
(12) Patent Application Publication
(10) Pub. No.: US 2010/0129470 A1
(43) Pub. Date: May 27, 2010

(54) METHOD OF TREATING BRAIN CANCER

(75) Inventor: MARK LAUGHLIN, Sunnyvale, CA (US)

Correspondence Address: Myriad PHARMACEUTICALS, Inc.;c/o CPA Global, P.O. Box 52050, Minneapolis, MN, 55402, US

(73) Assignee: Myriad Pharmaceuticals, Incorporated, Salt Lake City, UT (US)

(21) Appl. No.: 12/575,922

(22) Filed: Oct. 8, 2009

Related U.S. Application Data
(63) Continuation of application No. PCT/US2008/059905, filed on Apr. 10, 2008.
(60) Provisional application No. 60/910,975, filed on Apr. 10, 2007.

(51) Int. Cl. A61K 31/517; A61P 35/00; A61K 31/655; A61K 31/7048; A61K 31/704; A61K 31/519; A61K 31/7068; A61K 33/24

(52) U.S. Cl. 424/649; 514/266.4; 514/151; 514/27; 514/34; 514/249; 514/49

(57) ABSTRACT

Disclosed is (4-Methoxy-phenyl)-methyl-(2-methyl-quinazolin-4-yl)-amine hydrochloride effective as a cytotoxic agent. (4-Methoxy-phenyl)-methyl-(2-methyl-quinazolin-4-yl)-amine hydrochloride is useful in the treatment of a variety of clinical conditions in which uncontrolled growth and spread of abnormal cells occurs, and in particular to its use in treating brain cancer.

### CROSS REFERENCE TO RELATED APPLICATIONS

This application is a continuation of International Application No. PCT/US2008/059905, filed Apr. 10, 2008 and published as WO 2008/124822, which claims the benefit of U.S. Provisional Application Ser. No. 60/910,975, filed on Apr. 10, 2007; both of which are incorporated by reference herein in their entirety.

### FIELD OF THE INVENTION

This invention is in the field of medicinal chemistry. In particular, the invention relates to (4-Methoxy-phenyl)-methyl-(2-methyl-quinazolin-4-yl)-amine hydrochloride as a cytotoxic agent and use as a therapeutically effective anti-cancer agent.

### BACKGROUND OF THE INVENTION

Cancer is a common cause of death in the world; about 10 million new cases occur each year, and cancer is responsible for 12% of deaths worldwide, making cancer the third leading cause of death. World Health Organization, National Cancer Control Programmes: Policies and Managerial Guidelines (2d ed. 2002)

Despite advances in the field of cancer treatment, the leading therapies to date include surgery, radiation, and chemotherapy. Chemotherapeutic approaches are said to fight cancers that are metastasized or that are particularly aggressive. Most of the cancer chemotherapy agents currently in clinical use are cytotoxins. Cytotoxic agents work by damaging or killing cells that exhibit rapid growth. Ideal cytotoxic agents would have specificity for cancer and tumor cells, while not affecting normal cells. Unfortunately, none have been found and instead agents that target especially rapidly dividing cells (both tumor and normal) have been used.

Accordingly, materials that are cytotoxic to cancer cells while exerting only mild effects on normal cells are highly desirable. In fact, many recent studies have focused on developing alternative anticancer substances capable of specifically suppressing proliferation of tumor cells. Examples of such anticancer compounds may be found in International Pat. Publication No. WO 2005/003100. However, the safety of such compounds and amounts of such compounds that may be safely administered to an individual has not been known. Therefore, there remains a definite need in the art for the discovery of new effective chemotherapeutic agents and dosing ranges that can be administered safely.

### SUMMARY OF THE INVENTION

The present invention is related to the activity of (4-Methoxy-phenyl)-methyl-(2-methyl-quinazolin-4-yl)-amine hydrochloride, as a potent tubulin inhibitor and cytotoxic agent. (4-Methoxy-phenyl)-methyl-(2-methyl-quinazolin-4-yl)-amine hydrochloride is also known to achieve adequate concentration in the brain and CNS to be effective as treatment and/or prophylaxis for diseases and disorders of the brain and CNS, such as brain and spinal cord tumors.

It has been discovered that (4-Methoxy-phenyl)-methyl-(2-methyl-quinazolin-4-yl)-amine hydrochloride is tolerated in human patients at various concentrations and dosing levels. Accordingly, an aspect of the present invention is directed to the use of (4-Methoxy-phenyl)-methyl-(2-methyl-quinazolin-4-yl)-amine hydrochloride as therapy or prophylaxis for diseases and disorders of the brain and CNS at a dose of not more than about 4.5 mg/m². For example, the invention provides a method for treating cancers of the brain and CNS at a dose of between about 0.3 to about 3.3 mg/m², such as between about 2.1 mg/m² and about 3.3 mg/m². In particular examples, (4-Methoxy-phenyl)-methyl-(2-methyl-quinazolin-4-yl)-amine hydrochloride may be administered for treatment of cancers of the brain and CNS at a dose of between about 0.5 mg to about 15 mg, such as about 2 mg to about 10 mg, or about 4 mg to about 8 mg.

In certain aspects of the invention, the cancer treated by administration of (4-Methoxy-phenyl)-methyl-(2-methyl-quinazolin-4-yl)-amine hydrochloride is an anaplastic astrocytoma, glioblastoma, gliosarcoma, meningioma, or other mesenchymal tumor. Furthermore, the cancer treated by administration of 4-Methoxy-phenyl)-methyl-(2-methyl-quinazolin-4-yl)-amine hydrochloride may be recurrent or have one or more residual primary lesions after previous treatment.

Another aspect of the present invention relates to the administration of (4-Methoxy-phenyl)-methyl-(2-methyl-quinazolin-4-yl)-amine hydrochloride in combination with other known chemotherapeutic agents, including platinum compounds such as Paraplatin® (carboplatin) or Eloxatin® (oxaliplatin).

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

It is known that (4-Methoxy-phenyl)-methyl-(2-methyl-quinazolin-4-yl)-amine hydrochloride, is active as a potent tubulin inhibitor and cytotoxic agent. It is also known that (4-Methoxy-phenyl)-methyl-(2-methyl-quinazolin-4-yl)-amine hydrochloride is able to achieve adequate concentration in the brain and CNS to be effective as treatment and/or prophylaxis for diseases and disorders of the brain and CNS. In particular, (4-Methoxy-phenyl)-methyl-(2-methyl-quinazolin-4-yl)-amine hydrochloride is able to treat diseases of the brain and CNS that are responsive to therapy by inducing apoptosis, activating caspases, inhibiting tubulin and/or topoisomerase in the brain. Such diseases include, for example, brain and spinal cord tumors.

It has been discovered that (4-Methoxy-phenyl)-methyl-(2-methyl-quinazolin-4-yl)-amine hydrochloride is safely tolerated in human patients at various concentrations and dosing levels. In particular, two Phase 1 studies have been performed as open-label, dose-escalating, multiple-dose studies to define the safety, tolerability and phamacokinetics of weekly intravenous administration of (4-Methoxy-phenyl)-methyl-(2-methyl-quinazolin-4-yl)-amine hydrochloride. The results of such Phase 1 studies show that (4-Methoxy-phenyl)-methyl-(2-methyl-quinazolin-4-yl)-amine hydrochloride is safe and well tolerated in human subjects at various dosages.

Accordingly, an aspect of the present invention is directed to the use of (4-Methoxy-phenyl)-methyl-(2-methyl-quinazolin-4-yl)-amine hydrochloride as therapy or prophylaxis for diseases and disorders of the brain and CNS at a dose of not more than about 4.5 mg/m². In certain embodiments, (4-Methoxy-phenyl)-methyl-(2-methyl-quinazolin-4-yl)-amine hydrochloride is administered at a dose of not more than about 3.3 mg/m². In some embodiments, (4-Methoxy-phenyl)-methyl-(2-methyl-quinazolin-4-yl)-amine hydrochloride is administered at a dose of not more than about 2.7 mg/m². In further embodiments, (4-Methoxy-phenyl)-methyl-(2-methyl-quinazolin-4-yl)-amine hydrochloride is administered at a dose of not more than about 2.1 mg/m². In particular embodiments, the invention provides a method for treating cancers of the brain and CNS at a dose of between about 0.3 and 3.3 mg/m², such as between about 2.1 mg/m² and about 3.3 mg/m².

For example, (4-Methoxy-phenyl)-methyl-(2-methyl-quinazolin-4-yl)-amine hydrochloride may be administered for treatment of cancers of the brain and CNS at a dose of between about 0.5 mg to about 15 mg, such as about 2 mg to about 10 mg, or about 4 mg to about 8 mg. In certain embodiments, 4-Methoxy-phenyl)-methyl-(2-methyl-quinazolin-4-yl)-amine hydrochloride is administered at a dose of not more than about 10 mg, such as not more than about 8 mg or not more than about 6 mg. In additional embodiments, 4-Methoxy-phenyl)-methyl-(2-methyl-quinazolin-4-yl)-amine hydrochloride is administered at a dose of about 2, about 3, about 4, about 5, about 6, about 7, or about 8 mg.

There are various types of cancer found in the brain and CNS that may be treated according to the method of the current invention. Brain tumors can be generally classified as either primary brain tumors or metastatic brain tumors. Brain tumors are often further classified by cell type, morphology, cytogenetics, molecular genetics, immunologic markers, and/or a combination thereof. For example, brain tumors may be classified as neuroepithelial tumors (e.g. glial tumors, neuronal and mixed neuronal-glial tumors, and nonglial tumors), meningeal tumors, germ cell tumors, tumors of the sellar region, primary CNS lymphoma, tumors of peripheral nerves that affect the CNS, tumors of uncertain histogenesis, and metastatic tumors. A classification of brain tumors by The World Health Organization categorizes CNS tumors according to a malignancy scale based on histological features of the tumor (see Kleihues et al., Brain Pathol 3:255-268 (1993).

The most common types of primary brain tumors are anaplastic astrocytomas and glioblastomas, which account for approximately 38% of primary brain tumors; and meningiomas and other mesenchymal tumors, which account for approximately 27% of primary brain tumors. (see, Levin et al., Neoplasms of the central nervous system. In DeVita, et al., eds., Cancer: Principles and Practice of Oncology, Sixth Edition, Lippincott Williams & Wilkins, Philadelphia (2001), pp. 2100-2160). Other common primary brain tumors include pitutitary tumors, schwannomas, CNS lymphoma, oligodendrogliomas, ependymomas, low-grade astrocytomas, and medulloblastomas. Additional specific primary brain tumors include, astocytic tumors, pilocytic astrocytomas, diffuse astrocytomas, pleomorphic xanthoastrocytomas, subependymal giant cell astrocytomas, oligodendroglial tumors, olodendrogliomas, anaplastic oligodendrogliomas, oligoastrocytomas, anaplastic oligoastrocytomas, myxopapillary ependymomas, subependymomas, ependymomas, anaplastic ependymomas, astroblastomas, chordoid gliomas of the third ventricle, gliomatosis cerebris, glangliocytomas, desmoplastic infantile astrocytomas, desmoplastic infantile gangliogliomas, dysembryoplastic neuroepithelial tumors, central neurocytomas, cerebellar liponeurocytomas, paragangliomas, ependymoblastomas, medulloblastomas, supratentorial primitive neuroectodermal tumors, choroids plexus papilloma, pineocytomas, pineoblastomas, pineal parenchymal tumors of intermediate differentiation, hemangiopericytomas, melanocytic lesions, germ cell tumors, tumors of the sellar region, craniopharyngioma, capillary hemangioblastoma, and primary CNS lymphoma.

Metastatic brain tumors outnumber primary brain tumors by at least 10 to 1 and typically occur as a result of primary lung, breast, melanoma, or colon cancers metastasizing to the brain (Patchell R A, Cancer Treat. Rev. 29:533-540 (2003)). Cancers metastasizing to the brain result in multiple brain metastases in over 70% of cases (Patchell R A, Cancer Treat. Rev. 29:533-540 (2003)). And thus are not typically treated by surgery. However, chemotherapy is indicated to play a role in the treatment of patients with brain metastases from chemosensitive tumors (Patchell R A, Cancer Treat. Rev. 29:533-540 (2003). Thus, the therapeutic methods of the present invention for treating brain cancer, include treating primary brain neoplasms and brain metasases, by administering to an animal an effective amount of (4-Methoxy-phenyl)-methyl-(2-methyl-quinazolin-4-yl)-amine hydrochloride, or a pharmaceutically acceptable salt or prodrug thereof.

In one embodiment, the invention provides a method of reducing the size or slowing the growth of brain neoplasms. Reductions in size and/or growth of neoplasms may be measured by the Response Evaluation Criteria in Solid Tumors (RECIST) Guidelines (see Therasse et al. J. Nat. Cancer Institute 92:205-216 (2000), herein incorporated by reference in its entirety). For example, the method may reduce the average size of lesions in patients by about 30% or more as measured at four weeks post-treatment by identifying up to 5 lesions per organ and 10 lesions in total, and determining the reduction in length at the longest diameter of the lesion. In yet another embodiment, the invention provides a method for improving the survival of patients with or at risk of forming brain tumors.

In certain aspects of the invention, the cancer treated by administration of (4-Methoxy-phenyl)-methyl-(2-methyl-quinazolin-4-yl)-amine hydrochloride is an anaplastic astrocytoma, glioblastoma, gliosarcoma, meningioma, or other mesenchymal tumor. In specific embodiments, the cancer treated is glioblastoma. In some embodiments, the cancer treated by administration of 4-Methoxy-phenyl)-methyl-(2-methyl-quinazolin-4-yl)-amine hydrochloride may be recurrent or have one or more residual primary lesions after previous treatment, such as recurrent glioblastoma.

In specific embodiments, an anaplastic astrocytoma, glioblastoma, gliosarcoma, meningioma, or other mesenchymal tumor is treated by administering (4-Methoxy-phenyl)-methyl-(2-methyl-quinazolin-4-yl)-amine hydrochloride at a dose of not more than about 4.5 mg/m², such as not more than about 3.3 mg/m² or not more than about 2.1 mg/m². In certain embodiments, (4-Methoxy-phenyl)-methyl-(2-methyl-quinazolin-4-yl)-amine hydrochloride is administered to treat glioblastoma (such as recurrent glioblastoma) at a dose of not more than about 4.5 mg/m², such as not more than about 3.3 mg/m² or not more than about 2.1 mg/m². In some embodiments, (4-Methoxy-phenyl)-methyl-(2-methyl-quinazolin-4-yl)-amine hydrochloride is administered at a dose of not more than about 3.3 mg/m², such as between about 0.3 and 3.3 mg/m² or between about 2.1 mg/m² and about 3.3 mg/m² as therapy for an anaplastic astrocytoma, glioblastoma, gliosarcoma, meningioma, or other mesenchymal tumor. In further embodiments, (4-Methoxy-phenyl)-methyl-(2-methyl-quinazolin-4-yl)-amine hydrochloride is administered at a dose of not more than about 3.3 mg/m², such as between about 0.3 and 3.3 mg/m² or between about 2.1 mg/m² and about 3.3 mg/m² as therapy for glioblastoma (such as recurrent glioblastoma).

In some embodiments an anaplastic astrocytoma, glioblastoma, gliosarcoma, meningioma, or other mesenchymal tumor is treated by administering (4-Methoxy-phenyl)-methyl-(2-methyl-quinazolin-4-yl)-amine hydrochloride at a dose of between about 0.5 mg to about 15 mg, such as about 2 mg to about 10 mg, or about 4 mg to about 8 mg. In particular embodiments, a glioblastoma (such as recurrent glioblastoma) is treated by administering (4-Methoxy-phenyl)-methyl-(2-methyl-quinazolin-4-yl)-amine hydrochloride at a dose of between about 0.5 mg to about 15 mg, such as about 2 mg to about 10 mg, or about 4 mg to about 8 mg.

Another aspect of the present invention relates to the administration of (4-Methoxy-phenyl)-methyl-(2-methyl-quinazolin-4-yl)-amine hydrochloride in combination with other known chemotherapeutic agents. Chemotherapeutic agents useful in the present invention include nitrosoureas, such as Temodar® (temozolomide), dacarbazine, BCNU, and CCNU; taxanes, such as paclitaxel and docetaxel; vinka alkaloids, such as vincristine, vinblastine, and vinorelbine; topoisomerase inhibitors, such as etoposide, teniposide, Hycamtin® (topotecan), and Camptosar® (irinotecan); anthracyclines, such as doxorubicin, daunomycin, epirubicin, and idarubicin; antimetabolites, such as methotrexate, fluorouracil, cytarabine, Gemzar® (gemcitibine), and capecitibine; and platinum agents, such as cisplatin, Paraplatin® (carboplatin), and Eloxatin® (oxaliplatin). In certain embodiments, (4-Methoxy-phenyl)-methyl-(2-methyl-quinazolin-4-yl)-amine hydrochloride may be used as therapy in combination with one or more agents chosen from temozolomide, dacarbazine, BCNU, CCNU, vinorelbine, teniposide, irinotecan, daunomycin, idarubicin, cytarabine, gemcitibine, capecitibine, and oxaliplatin. In certain embodiments, 4-Methoxy-phenyl)-methyl-(2-methyl-quinazolin-4-yl)-amine hydrochloride may be administered with one or more platinum compounds, such as carboplatin. In particular embodiments, 4-Methoxy-phenyl)-methyl-(2-methyl-quinazolin-4-yl)-amine hydrochloride may be administered with one or more platinum compounds, such as oxaliplatin.

In specific embodiments, 4-Methoxy-phenyl)-methyl-(2-methyl-quinazolin-4-yl)-amine hydrochloride is administered at a dose of not more than about 4.5 mg/m², such as not more than about 3.3 mg/m² or not more than about 2.1 mg/m² in combination with other known chemotherapeutic agents such as Temodar® (temozolomide), dacarbazine, BCNU, and CCNU; taxanes, such as paclitaxel and docetaxel; vinka alkaloids, such as vincristine, vinblastine, and vinorelbine; topoisomerase inhibitors, such as etoposide, teniposide, Hycamtin® (topotecan), and Camptosar® (irinotecan); anthracyclines, such as doxorubicin, daunomycin, epirubicin, and idarubicin; antimetabolites, such as methotrexate, fluorouracil, cytarabine, Gemzar® (gemcitibine), and capecitibine; and platinum agents, such as cisplatin, carboplatin, and Eloxatin® (oxaliplatin).

In certain embodiments, 4-Methoxy-phenyl)-methyl-(2-methyl-quinazolin-4-yl)-amine hydrochloride is administered at a dose of not more than about 4.5 mg/m², such as not more than about 3.3 mg/m² or not more than about 2.1 mg/m² in combination with one or more chemotherapeutic agents chosen from temozolomide, dacarbazine, BCNU, CCNU, vinorelbine, teniposide, irinotecan, daunomycin, idarubicin, cytarabine, gemcitibine, capecitibine, carboplatin, and oxaliplatin. In certain embodiments, 4-Methoxy-phenyl)-methyl-(2-methyl-quinazolin-4-yl)-amine hydrochloride is administered at a dose of not more than about 4.5 mg/m², such as not more than about 3.3 mg/m² or not more than about 2.1 mg/m² in combination with carboplatin. In particular embodiments, 4-Methoxy-phenyl)-methyl-(2-methyl-quinazolin-4-yl)-amine hydrochloride is administered at a dose of not more than about 4.5 mg/m², such as not more than about 3.3 mg/m² or not more than about 2.1 mg/m² in combination with oxaliplatin.

For example (4-Methoxy-phenyl)-methyl-(2-methyl-quinazolin-4-yl)-amine hydrochloride may be administered at a dose of between about 0.5 mg to about 15 mg, such as about 2 mg to about 10 mg, or about 4 mg to about 8 mg in combination with one or more chemotherapeutic agents chosen from temozolomide, dacarbazine, BCNU, CCNU, vinorelbine, teniposide, irinotecan, daunomycin, idarubicin, cytarabine, gemcitibine, capecitibine, carboplatin, and oxaliplatin. In certain embodiments, 4-Methoxy-phenyl)-methyl-(2-methyl-quinazolin-4-yl)-amine hydrochloride is administered at a dose of not more than about 10 mg, such as not more than about 8 mg or not more than about 6 mg in combination with carboplatin. In particular embodiments, 4-Methoxy-phenyl)-methyl-(2-methyl-quinazolin-4-yl)-amine hydrochloride is administered at a dose of not more than about 10 mg, such as not more than about 8 mg or not more than about 6 mg in combination with oxaliplatin.

In some embodiments, an anaplastic astrocytoma, gliosarcoma, meningioma, or other mesenchymal tumor (for example, glioblastoma or recurrent glioblastoma) is treated with 4-Methoxy-phenyl)-methyl-(2-methyl-quinazolin-4-yl)-amine hydrochloride at a dose of not more than about 4.5 mg/m², such as not more than about 3.3 mg/m² or not more than about 2.1 mg/m² in combination with one or more chemotherapeutic agents chosen from temozolomide, dacarbazine, BCNU, CCNU, vinorelbine, teniposide, irinotecan, daunomycin, idarubicin, cytarabine, gemcitibine, capecitibine, carboplatin, and oxaliplatin. In certain embodiments, an anaplastic astrocytoma, gliosarcoma, meningioma, or other mesenchymal tumor (for example, glioblastoma or recurrent glioblastoma) is treated with 4-Methoxy-phenyl)-methyl-(2-methyl-quinazolin-4-yl)-amine hydrochloride at a dose of not more than about 4.5 mg/m², such as not more than about 3.3 mg/m² or not more than about 2.1 mg/m² in combination with carboplatin. In further embodiments, an anaplastic astrocytoma, gliosarcoma, meningioma, or other mesenchymal tumor (for example, glioblastoma or recurrent glioblastoma) is treated with 4-Methoxy-phenyl)-methyl-(2-methyl-quinazolin-4-yl)-amine hydrochloride at a dose of not more than about 4.5 mg/m², such as not more than about 3.3 mg/m² or not more than about 2.1 mg/m² in combination with oxaliplatin.

In practicing the methods of the present invention, (4-Methoxy-phenyl)-methyl-(2-methyl-quinazolin-4-yl)-amine hydrochloride may be administered together with at least one known chemotherapeutic agent as part of a unitary pharmaceutical composition. Alternatively, (4-Methoxy-phenyl)-methyl-(2-methyl-quinazolin-4-yl)-amine hydrochloride may be administered apart from at least one known cancer chemotherapeutic agent. In one embodiment, (4-Methoxy-phenyl)-methyl-(2-methyl-quinazolin-4-yl)-amine hydrochloride and at least one known cancer chemotherapeutic agent are administered substantially simultaneously, i.e. the compounds are administered at the same time or one after the other, so long as the compounds reach therapeutic levels in the blood at the same time. On another embodiment, the compound of the invention and at least one known cancer chemotherapeutic agent are administered according to their individual dose schedule, so long as the compounds reach therapeutic levels in the blood.

In particular embodiments, (4-Methoxy-phenyl)-methyl-(2-methyl-quinazolin-4-yl)-amine hydrochloride is administered with carboplatin. Carboplatin may be administered at various doses, such as doses of not more than about 700 mg, such as between about 100 mg to about 700 mg, between about 200 mg to about 600 mg, or between about 300 to about 500 mg, before, after or concurrently with administration of (4-Methoxy-phenyl)-methyl-(2-methyl-quinazolin-4-yl)-amine hydrochloride. For example, carboplatin may be administered to a subject at a dose of about 100, about 200, about 300, about 400, about 500, about 600, or about 700 mg. Carboplatin may be administered at a dose that provides the subject an AUC (area under curve) of not more than about 6 (mg/mL)*(min), such as from about 3 (mg/mL)*(min) to about 6 (mg/mL)*(min), or from about 4 (mg/mL)*(min) to about 6 (mg/mL)*(min), before, after or concurrently with administration of (4-Methoxy-phenyl)-methyl-(2-methyl-quinazolin-4-yl)-amine hydrochloride.

In particular embodiments, (4-Methoxy-phenyl)-methyl-(2-methyl-quinazolin-4-yl)-amine hydrochloride is administered with oxaliplatin. Oxaliplatin may be administered at various doses, such as doses of not more than about 150 mg/m², such as from about 25 mg/m² to about 100 mg/m² before, after or concurrently with administration of (4-Methoxy-phenyl)-methyl-(2-methyl-quinazolin-4-yl)-amine hydrochloride. Oxaliplatin may be also be administered at a dose of not more than about 85 mg/m², such as from about 55 mg/m² to about 85 mg/m² before, after or concurrently with administration of (4-Methoxy-phenyl)-methyl-(2-methyl-quinazolin-4-yl)-amine hydrochloride.

The dosage of active compound(s) administered is dependent on the body weight, age, individual condition, and on the form of administration. The active compound(s) may be administered to a subject over various time frames and for varying lengths. For example, active compounds that are infused may be administered through an infusion process that last 0.5, 1, 2, 3, 4, or 8 hours. Additionally, the active compounds may be administered daily, weekly, monthly, or according to various schedules such as cycles of once a week for three weeks followed by a week of no administration. For example, the active compounds(s), such as (4-Methoxy-phenyl)-methyl-(2-methyl-quinazolin-4-yl)-amine hydrochloride and carboplatin may be administered once every two weeks on a six week cycle. In another example, the active compounds, such as (4-Methoxy-phenyl)-methyl-(2-methyl-quinazolin-4-yl)-amine hydrochloride may be administered on an eight week schedule with (4-Methoxy-phenyl)-methyl-(2-methyl-quinazolin-4-yl)-amine hydrochloride administered once every week for six weeks followed by no administration for two weeks.

Compounds used in practicing the present invention can be prepared by a variety of art known procedures. For example, in practicing the present invention, (4-Methoxy-phenyl)-methyl-(2-methyl-quinazolin-4-yl)-amine hydrochloride may be prepared using methods known to those skilled in the art. Specifically, (4-Methoxy-phenyl)-methyl-(2-methyl-quinazolin-4-yl)-amine hydrochloride may be prepared according to International Pat. Publication No. WO 2005/003100 and as illustrated by the exemplary reaction in Scheme 1.

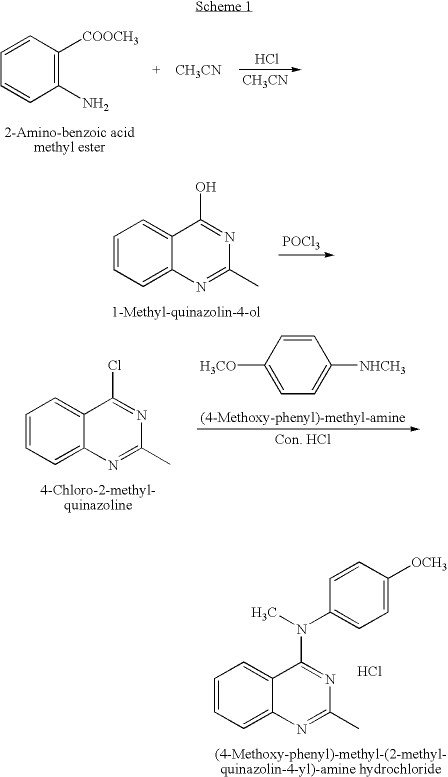

In addition, many of the compounds are commercially available from a variety of sources. For example, carboplatin is available from Bristol-Myers Squibb Company (New York, N.Y.), oxaliplatin is available from Sanofi-Aventis (Paris, France), and temozolomide is available from Schering-Plough (Kenilworth, N.J.).

The therapeutic methods of present invention also include methods comprising administering to an animal an effective amount of a compound, or a pharmaceutically acceptable salt, acid or base of (4-Methoxy-phenyl)-methyl-(2-methyl-quinazolin-4-yl)-amine hydrochloride. In one embodiment, a pharmaceutical composition comprising (4-Methoxy-phenyl)-methyl-(2-methyl-quinazolin-4-yl)-amine hydrochloride, or a pharmaceutically acceptable salt, acid, or base of said compound, in combination with a pharmaceutically acceptable vehicle is administered. Examples of pharmaceutically acceptable addition salts for (4-Methoxy-phenyl)-methyl-(2-methyl-quinazolin-4-yl)-amine hydrochloride, (or base thereof) include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine. The present invention also includes methods comprising administering to an animal an effective amount of (4-Methoxy-phenyl)-methyl-(2-methyl-quinazolin-4-yl)-amine hydrochloride, or a pharmaceutically acceptable salt or prodrug thereof, and one or more liquid diluents. Such compositions include compositions disclosed in PCT Pub. No. WO 2006/138608, and may be manufactured according to the methods disclosed therein, the relevant portions of which are incorporated herein by reference.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

### Preparation of (4-Methoxy-phenyl)-methyl-(2-methyl-quinazolin-4-yl)-amine hydrochloride

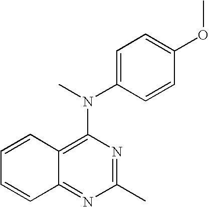

### (4-Methoxy-phenyl)-methyl-(2-methyl-quinazolin-4-yl)-amine hydrochloride

a) 4-Chloro-2-methyl-quinazoline: A stirred suspension of 2-methyl-4(3H)-quinazolinone (5 g, 31.2 mmol) in POCl₃ (100 mL) was heated at 120° C. for 3 h. The excess POCl₃ was removed under vacuum, then to the residue was added crushed ice and 200 mL of saturated NaHCO₃, and the mixture was extracted with ethyl acetate (200 mL×2). The combined extracts were washed with water, saturated NaCl, dried over anhydrous MgSO₄, filtered and concentrated. The crude product was purified by column chromatography (5-8% ethyl acetate/hexane) to give the title compound (2.5 g, 14.0 mmol, 45%). ¹H NMR (CDCl₃): 8.21-8.25 (m, 1H), 7.89-7.99 (m, 2H), 7.66 (ddd, 1H, J=1.8, 6.6, 8.7), 2.87 (s, 3H).

b) (4-Methoxy-phenyl)-methyl-(2-methyl-quinazolin-4-yl)-amine hydrochloride: The title compound was prepared from 4-chloro-2-methyl-quinazoline (2.31 g, 12.9 mmol) and (4-methoxy phenyl)-methyl-amine (2.0 g, 14.6 mmol) by a procedure similar to example 1b and was isolated as solids (2.90 g, 9.18 mmol, 71%). ¹H NMR (CDCl₃): 8.53 (dd, 1H, J=0.6, 8.1), 7.7 (ddd, 1H, J=1.2, 7.2, 8.4), 7.22 (m, 2H), 7.13 (ddd, 1H, J=1.2, 7.2, 8.7), 7.05 (m, 2H), 6.76 (d, 1H, J=8.7), 3.91 (s, 3H), 3.78 (s, 3H), 2.96 (s, 3H).

### Example 2

### Pharmaceutical Composition

A pharmaceutical composition is prepared by combining and mixing 100 grams of (4-Methoxy-phenyl)-methyl-(2-methyl-quinazolin-4-yl)-amine hydrochloride and 1 gram of BHT and dissolving into 10 liters of D5W with the pH adjusted to pH=5 with hydrochloric acid. This solution is sterile filtered using a 0.2 μm Teflon filter (PTFE).

### Example 3

### Pharmaceutical Composition

A pharmaceutical composition was formed by dissolving 300.1 grams (4-Methoxy-phenyl)-methyl-(2-methyl-quinazolin-4-yl)-amine hydrochloride into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 4

### Pharmaceutical Composition

A pharmaceutical composition was formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 5

### Pharmaceutical Composition

A pharmaceutical composition is formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 11.652 kg viscosity reducing agent (ethanol 190 proof), and 2 kg WFI (water for injection). This solution is sterile filtered through a 0.2 μm Millipore Durapore filter (PVDF), and packaged into 10 ml sterile glass vials.

### Example 6

### Method of Administration

About 0.01 ml to about 50 ml of the pharmaceutical composition of Example 5 is accurately measured and then added to an i.v. bag containing about 100 ml to about 1000 ml of sterile dextrose 5% in water (D5W). The amount of pharmaceutical composition and D5W used varies according to the desired therapeutic dose and size of the patient. The resulting mixture is then parenterally infused into the patient.

### Example 7

### Phase I Clinical Trial of Administration of (4-Methoxy-phenyl)-methyl-(2-methyl-quinazolin-4-yl)-amine hydrochloride for Subjects with Refractory Solid Tumors

An open-label, dose-escalating, multiple-dose study to define the safety, tolerability and phamacokinetics of weekly intravenous administration of (4-Methoxy-phenyl)-methyl-(2-methyl-quinazolin-4-yl)-amine hydrochloride was performed. A dosing schedule (each 4 week cycle) was performed for (4-Methoxy-phenyl)-methyl-(2-methyl-quinazolin-4-yl)-amine hydrochloride weekly for 3 weeks with no infusion on the fourth week of each cycle. Subjects with refractory solid tumors were enrolled in cohorts of 3. During Cycle 1, subjects were hospitalized during each infusion of (4-Methoxy-phenyl)-methyl-(2-methyl-quinazolin-4-yl)-amine hydrochloride and remained for observation and safety evaluation for approximately 24 hours following the end of the infusion. All subject had continuous telemetry for 2 hours prior to infusion, for 1-2 hour infusion and for 3 hours after the end of the infusion. Any clinically significant electrocardiographic (ECG) wave form abnormality was recorded and prolongation of the monitoring period extended at the discretion of the principal investigator.

Electrocardiograms were obtrained prior to starting the infusion and within 30 minutes of the end of infusion for each infusino of the first cycle. Electrocardiograms on Day 1 were obtained in triplicate 5 minutes apart.

Neurocognitive assessments were made by administration of the Mini-Mental State Examination (MMSE), the Hopkins Verbal Learning and timed Grooved Pegboard tests before administration of the intravenous infusion and approximately 24 hours of the infusion at each weekly administration of the first cycle.

On days 1, 8, and 15 of each cycle, vital signs were obtained prior to the first dose, at 15, 30, and 60 minutes after the initiation of the infusion, and at 0.5, 1, 1.5, 2, and 4 hours after the end of the intravenous infusion. Vital signs at all time points beyond the start of the intravenous infusion included heart rate, blood pressure and respirations. Temperature ws measured at the end of the infusion and 4 hours later.

Individual subjects were allowed to continue on repeated weekly×3 administrations every 28 days with no dose increase provided there was no unacceptable toxicity or disease progression.

Tumor response was evaluated by response evaluation criteria in solid tumors (RECIST) criteria. To prevent sever hypersensitivity reactions due to Cremophor® EL, subjects were premedicated with oral dexamethasone (20 mg) administered approximately 12 and 6 hours before the intrvenous infusion with (4-Methoxy-phenyl)-methyl-(2-methyl-quinazolin-4-yl)-amine hydrochloride, diphenhydramine (50 mg) or its equivalent administered intravenously 30-60 minutes before (4-Methoxy-phenyl)-methyl-(2-methyl-quinazolin-4-yl)-amine hydrochloride, and cimetidine (300 mg) or ranitidine (50 mg) administered intravenously 30-60 minutes before (4-Methoxy-phenyl)-methyl-(2-methyl-quinazolin-4-yl)-amine hydrochloride.

Dose escalation of subjects proceeded sequentially as presented in Table 1 below:

TABLE 1

| Cohort Number | Dose level (modified Fibonacci series) |
|---|---|
| Cohort 1 | Dose 1 = 0.3 mg/m² |
| Cohort 2 | Dose 2 = 0.6 mg/m² |
| Cohort 3 | Dose 3 = 1.0 mg/m² |
| Cohort 4 | Dose 4 = 1.5 mg/m² |
| Cohort 5 | Dose 5 = 2.1 mg/m² |
| Cohort 6 | Dose 6 = 2.7 mg/m² |
| Cohort 7 | Dose 7 = 3.3 mg/m² |

The results of the Phase 1 Trial show that there is no evidence of cytotoxity peripherally at the administered doses. There were incidences of intratumor bleeding and the dose limiting toxicity was demonstrated to be vascular in nature, manifested by an acute coronary syndrome. There were no significant effects on cardiac conduction (PR, QRS or QTc) but there was a dose-related increase in systolic blood pressure and occasional episodes of bradycardia. Accordingly, (4-Methoxy-phenyl)-methyl-(2-methyl-quinazolin-4-yl)-amine hydrochloride is thus shown to be safe and tolerable.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating cancer of the brain or central nervous system in a mammal in need of such treatment, comprising administering to the mammal an effective amount of not more than about 4.5 mg/m² of (4-Methoxy-phenyl)-methyl-(2-methyl-quinazolin-4-yl)-amine hydrochloride, a molar equivalent of the free acid thereof, or a molar equivalent of another pharmaceutically acceptable salt of the free acid thereof.

2. The method of claim 1, wherein the cancer is an anaplastic astrocytoma, glioblastoma, gliosarcoma, meningioma, or other mesenchymal tumor.

3. The method of claim 1, wherein the cancer is recurrent or has one or more residual primary lesions after previous treatment.

4. The method of claim 1, wherein the effective amount of (4-Methoxy-phenyl)-methyl-(2-methyl-quinazolin-4-yl)-amine hydrochloride is administered at a dose of not more than about 3.3 mg/m².

5. The method of claim 1, wherein the effective amount of (4-Methoxy-phenyl)-methyl-(2-methyl-quinazolin-4-yl)-amine hydrochloride is administered at a dose of not more than about 2.7 mg/m².

6. The method of claim 1, wherein the effective amount of (4-Methoxy-phenyl)-methyl-(2-methyl-quinazolin-4-yl)-amine hydrochloride is administered at a dose of not more than about 2.1 mg/m².

7. The method of claim 1, wherein the mammal is also administered an effective amount of one or more other chemotherapeutic agents.

8. The method of claim 7, wherein one or more of the other chemotherapeutic agents is chosen from temozolomide, dacarbazine, BCNU, CCNU, paclitaxel, docetaxel, vincristine, vinblastine, vinorelbine, etoposide, teniposide, topotecan, irinotecan, doxorubicin, daunomycin, epirubicin, idarubicin, methotrexate, fluorouracil, cytarabine, gemcitibine, capecitibine, cisplatin, carboplatin, and oxaliplatin.

9. The method of claim 7, wherein one or more of the other chemotherapeutic agents is carboplatin.

10. The method of claim 9, wherein the effective amount of carboplatin is administered at a dose that provides the subject an AUC of not more than about 6 (mg/mL)*(min).

11. The method of claim 9, wherein
(a) (4-Methoxy-phenyl)-methyl-(2-methyl-quinazolin-4-yl)-amine hydrochloride is administered at a dose of between about 2.1 mg/m² and about 3.3 mg/m², and
(b) carboplatin is administered at a dose that provides the subject an AUC of between about 4 (mg/mL)*(min) and about 6 (mg/mL)*(min).

12. The method of claim 9, wherein the effective amount of carboplatin is administered at a dose of not more than 700 mg.

13. The method of claim 9, wherein the effective amount of carboplatin is administered at a dose of from about 100 mg to about 700 mg.

14. The method of claim 9, wherein the effective amount of carboplatin is administered at a dose of from about 300 mg to about 500 mg.

15. The method of claim 1, wherein the cancer of the brain comprises a primary brain tumor.

16. The method of claim 1, wherein the cancer of the brain comprises a metastatic brain tumor.

17. The method of claim 16, wherein the metastatic brain tumor metastasized from primary lung, breast, or colon cancer.

18. The method of claim 1, wherein the mammal is a human.

19. The method of claim 1, wherein administration occurs weekly.

20. A method of reducing the size of, or slowing the growth of, brain neoplasms in a mammal in need of such treatment, comprising administering to the mammal an effective amount of not more than about 4.5 mg/m² of (4-Methoxy-phenyl)-methyl-(2-methyl-quinazolin-4-yl)-amine hydrochloride, a molar equivalent of the free acid thereof, or a molar equivalent of another pharmaceutically acceptable salt of the free acid thereof.
